(19) United States
(12) Patent Application Publication
(10) Pub. No.: US 2017/0112619 A1
(43) Pub. Date: Apr. 27, 2017

(54) Systems and Methods of Sealing a Deployed Valve Component

(71) Applicant: Medtronic Vascular, Inc., Santa Rosa, CA (US)

(72) Inventors: Donna Curley, Ballybrit (IE); Marc Anderson, Ballybrit (IE)

(21) Appl. No.: 14/919,771

(22) Filed: Oct. 22, 2015

(51) Int. Cl. A61F 2/24

(52) U.S. Cl. CPC A61F 2/2409; A61F 2/2418; A61F 2/2433; A61F 2/243; A61F 2220/0016

(57) ABSTRACT

A sealing system for sealing a valve component in a radially expanded deployed configuration to a wall of a native valve includes a valve component and an expandable sealing ring. The valve component has an inflow portion and outflow portion and includes a frame and a prosthetic valve coupled to the frame. The frame defines a central passage with the prosthetic valve disposed therein. The expandable sealing ring is configured to be inserted in a compressed configuration within the central passage of the valve component with the valve component in the radially expanded deployed configuration. The expandable sealing ring is configured to be radially expanded to apply a radially outward force to the valve component, forcing the frame radially outward toward the wall of the native valve.

### FIELD OF THE INVENTION

The present invention relates to systems and methods for sealing a percutaneously implanted valve component including a prosthetic valve. More particularly, it relates to the systems and methods for sealing a deployed valve component via transcatheter implantation of a sealing ring.

### BACKGROUND

Heart valves are sometimes damaged by disease or by aging, resulting in problems with the proper functioning of the valve. Heart valve replacement has become a routine surgical procedure for patients suffering from valve dysfunctions. Traditional open surgery inflicts significant patient trauma and discomfort, requires extensive recuperation times, and may result in life-threatening complications.

To address these concerns, efforts have been made to perform cardiac valve replacements using minimally-invasive techniques. In these methods, laparoscopic instruments are employed to make small openings through the patient's ribs to provide access to the heart. While considerable effort has been devoted to such techniques, widespread acceptance has been limited by the clinician's ability to access only certain regions of the heart using laparoscopic instruments.

Still other efforts have been focused upon percutaneous transcatheter (or transluminal) delivery of replacement cardiac valves to solve the problems presented by traditional open surgery and minimally-invasive surgical methods. In such methods, a valve component including a prosthetic valve is compacted for delivery in a catheter and then advanced, for example through an opening in the native vasculature, and through to the heart, where the valve component is then deployed in a valve annulus (e.g., the aortic valve annulus).

Various types and configurations of prosthetic valves and valve components are available for percutaneous valve replacement procedures. In general, prosthetic valve designs for a heart attempt to replicate the function of the valve being replaced and thus will include valve leaflet-like structures. Prosthetic valves are generally formed by attaching a bioprosthetic valve to a frame made of a wire or a network of wires, creating a valve component. Such valve components can be contracted radially to introduce valve component into the body of the patient percutaneously through a catheter. The valve component can be deployed by radially expanding it once positioned at a desired target site.

In some patients, a wall of the native valve at the target site may be misshapen or heavily calcified. In such cases, the radial expansion of the valve component may not conform to the shape of the wall of the native valve. If the deployed valve component is not 100% coapted to the wall of the native valve, paravalvular leakage (PVL), a serious post surgical complication may arise.

Accordingly, there is a need for a system and method of sealing a valve component to the wall of the native valve after valve component implantation via transcatheter delivery devices and methods.

### SUMMARY OF INVENTION

Embodiments hereof relate to a sealing system for sealing a valve component in a radially expanded deployed configuration to a wall of the native valve. The sealing system includes the valve component and an expandable sealing ring. The valve component includes a frame and a prosthetic valve coupled to the frame. The valve component has an inflow portion and outflow portion. The frame defines a central passage with the prosthetic valve disposed therein. The expandable sealing ring is configured to be inserted in a compressed configuration within the central passage with the valve component in the radially expanded deployed configuration. The expandable sealing ring is configured to be radially expanded to apply a radially outward force to the valve component.

Embodiments hereof also relate to a method remodeling a valvular prosthesis. The valvular prosthesis includes a frame and a prosthetic valve coupled to the frame. The method includes advancing an expandable ring in a radially compressed configuration to a location within the frame of the valvular prosthesis with the frame in a radially expanded configuration within a native valve. The method further includes expanding the expandable ring to a radially expanded configuration such that the expandable ring forces the frame radially outwardly.

### BRIEF DESCRIPTION OF DRAWINGS

FIG. 1 is a cutaway side view illustration of a valve component deployed within a native artery.

FIG. 2A is a side perspective illustration of a sealing system according to an embodiment hereof.

FIG. 2B is a bottom view illustration of the sealing system of FIG. 2A.

FIG. 3A is a side perspective illustration of an expandable sealing ring of the sealing system of FIG. 2A.

FIG. 3B is a top or bottom view illustration of the expandable sealing ring of FIG. 3A.

FIG. 3C is a top or bottom view illustration an expandable sealing ring including a sealing material disposed around an outer surface thereof.

FIG. 4A is side perspective illustration of a sealing system according to another embodiment hereof.

FIG. 4B is a top view illustration of the sealing system of FIG. 4A.

FIG. 5A is a side perspective illustration a sealing system according to another embodiment hereof.

FIG. 5B is a perspective view illustration of an alternative expandable sealing ring for use with the sealing system of FIG. 5A.

FIG. 6 is a side perspective illustration a sealing system according to another embodiment hereof.

FIG. 7A is a side perspective illustration a sealing system according to another embodiment hereof.

FIG. 7B is a bottom view illustration of the sealing system of FIG. 7A.

FIG. 8A is a perspective view illustration of an expandable sealing ring of the sealing system of FIG. 7A.

FIG. 8B is a top or bottom view illustration of the expandable sealing ring of FIG. 8A.

FIG. 9 is a top view illustration of an expandable sealing ring according to another embodiment hereof.

FIG. 10A is a side perspective illustration a sealing system according to another embodiment hereof.

FIG. 10B is a top view illustration of the sealing system of FIG. 11A.

FIG. 11 is a top view illustration of an alternative embodiment of the sealing system of FIG. 10A.

FIGS. 12A, 12B, 13A, 13B, 14A, and 14B are simplified illustrations of a method of sealing a valve component in a radially expanded configuration to a wall of a native valve.

FIGS. 15A, 15B, 16A, 16B, 17A, 17B are simplified illustrations of a method of sealing a valve component in a radially expanded configuration to a wall of a native valve according to another embodiment hereof.

FIGS. 18A-18B are simplified illustrations of the method of FIGS. 15A-17B utilizing the sealing ring of FIG. 9.

FIGS. 19A, 19B, 20A, 20B, 21A, and 21B are simplified illustrations of a method of sealing a valve component in a radially expanded configuration to a wall of a native valve using the sealing system of FIGS. 10A-10B.

### DETAILED DESCRIPTION

Specific embodiments of the present invention are now described with reference to the figures, wherein like reference numbers indicate identical or functionally similar elements. The terms “distal” and “proximal”, when used in the following description to refer to a catheter or delivery device, are with respect to a position or direction relative to the treating clinician. Thus, “distal” and “distally” refer to positions distant from, or in a direction away from, the clinician and “proximal” and “proximally” refer to positions near, or in a direction toward, the clinician. When the terms “distal” and “proximal” are used in the following description to refer to a device implanted into a native artery, such as a valve component, they are used with reference to the direction of blood flow from the heart. Thus “distal” and “distally” refer to positions in a downstream direction with respect to the direction of blood flow and “proximal” and “proximally” refer to positions in an upstream direction with respect to the direction of blood flow.

The following detailed description is merely exemplary in nature and is not intended to limit the invention or the application and uses of the invention. Although the description of the invention is in the context of transcatheter aortic valve sealing systems, the invention may also be used in other body passageways where it is deemed useful. Furthermore, there is no intention to be bound by any expressed or implied theory presented in the preceding technical field, background, brief summary, or the following detailed description.

As referred to herein, a valve component used in accordance with and/or as part of the various systems, devices, and methods of the present disclosure may include a wide variety of different configurations, such as a bioprosthetic heart valve having tissue leaflets or a synthetic heart valve having polymeric, metallic, or tissue-engineered leaflets, and can be specifically configured for replacing any heart valve.

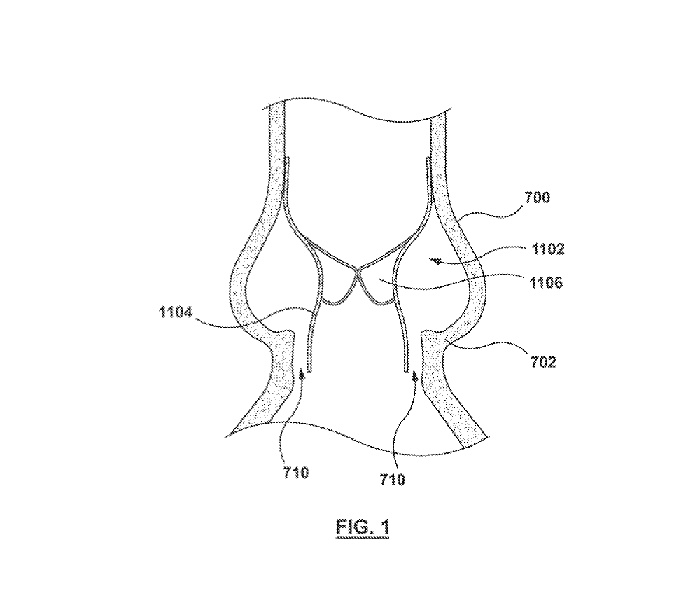

In some patients, the radial expansion of a valve component 1102, including a frame 1104 and a prosthetic valve 1106, as shown in FIG. 1, may not conform to the shape of the wall of the native valve 700. This situation may occur when the wall of the native valve 700 is misshapen or heavily calcified. In such cases where the deployed valve component 1104 is not 100% coapted to the wall of the native valve 700, paravalvular leakage (PVL) may occur between the valve component 1104 and the wall of the native valve. In FIG. 1, voids 710 are shown between the annulus 702 and the valve component 1104. However, this is not meant to be limiting, and such voids may occur between the valve component and the wall of the sinus, or between the valve component and the walls of the sinotubular junction or the ascending aorta. The phrase “wall of the native valve”, as used herein, means the walls surrounding the native valve, including walls slightly downstream and upstream of the native valve. Thus, for example, and not by way of limitation, the “wall of the native valve” for the aortic valve would include the annulus, the wall of the sinuses, the sinotubular junction, and the wall of the ascending aorta.

Embodiments hereof are related to a sealing system including a valve component and an expandable sealing ring. The term valve component, described in more detail below, may also be referred to as a valve prosthesis or valvular prosthesis, or other terms known to those skilled in the art.

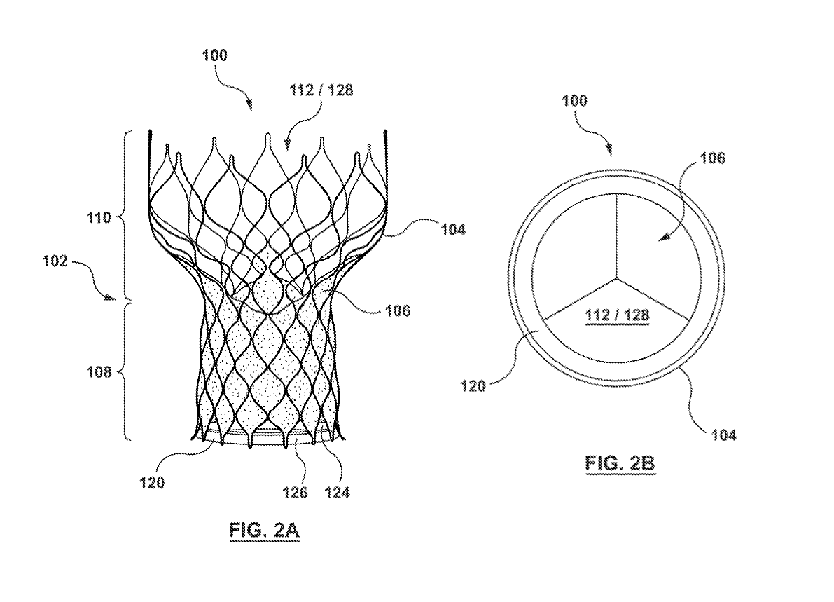

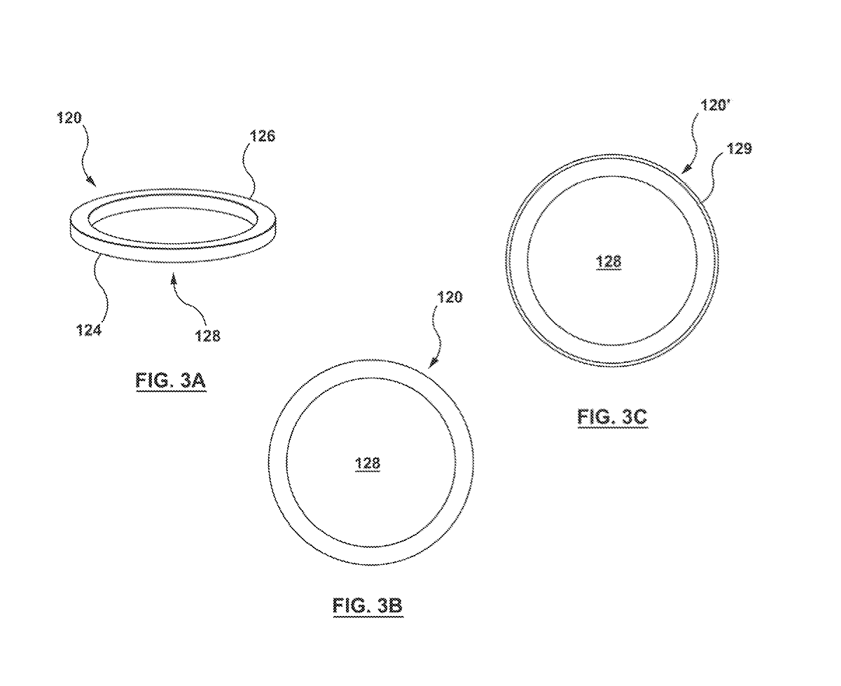

In an embodiment shown in FIGS. 2A, 2B, 3A, and 3B, a sealing system 100 includes a valve component 102 and an expandable sealing ring 120. Sealing system 100 may also be referred to as a device for remodeling a valvular prosthesis. In FIG. 2A, valve component 102 is in a radially expanded configuration.

Valve component 102 includes a frame 104 and a prosthetic valve 106. Valve component 102 may be a conventional valve prosthesis similar to the Medtronic CoreValve® transcatheter aortic valve replacement valve prosthesis and as described in U.S. Pat. No. 7,914,569 to Nguyen et al. (hereinafter “the '569 patent”), which is incorporated by reference herein in its entirety.

Frame 104 is a support structure that comprises a number of struts or wire portions arranged relative to each other to provide a desired compressibility and strength to prosthetic valve 106. Frame 104 is a stent structure as is known in the art. Frame 104 may be self-expandable, balloon-expandable, or otherwise mechanically expandable. Frame 104 may be any stent structure suitable for use with a prosthetic valve. For example, and not by way of limitation, frame 104 may be similar to the stent structures described in the '569 patent and U.S. Pat. No. 7,740,655 to Birdsall, which is incorporated by reference herein. Frame 104 is a generally tubular structure and defines a central passage 112.

Prosthetic valve 106 is coupled to and disposed within frame 104. Prosthetic valve 106 preferably includes individual leaflets formed from a natural or man-made material, including but not limited to, mammalian tissue, such as porcine, equine or bovine pericardium, or a synthetic or polymeric material. Prosthetic valve 106 may also include a skirt (not shown) affixed to frame 104, the leaflets of prosthetic valve 106 may be attached are attached along their bases to the skirt, for example, using sutures or a suitable biocompatible adhesive. Adjoining pairs of the leaflets are attached to one another at their lateral ends to form commissures (not shown), with free edges of the leaflets forming coaptation edges that meet in an area of coaptation, as described in the '569 patent.

Valve component 102 has an inflow portion 108 at a proximal end of valve component 102, and an outflow portion 110 at a distal end of valve component 102, as shown in FIG. 2A.

Expandable sealing ring 120 is a generally annular ring having a longitudinal first end 124 and a longitudinal second end 126 opposite first end 124, as shown in FIGS. 3A and 3B. Expandable sealing ring 120 has a compressed configuration for delivery to a treatment site and an expanded configuration when deployed. Expandable sealing ring 120 may be self-expanding, balloon expandable, or otherwise mechanically expandable. In the expanded configuration, expandable sealing ring 120 may have a diameter in the range of 18 to 29 millimeters for use in an aortic annulus. However, it is recognized that expandable sealing ring 120 may have a smaller or larger expanded diameter depending on the application. Further, the unrestrained expanded diameter of expandable sealing ring 120 is generally about 2-6 millimeters larger than the diameter of the location in which expandable sealing ring 120 is to be installed, in order to create opposing radial forces between the outward radial force of expandable sealing ring 120 against inward resisting forces of the wall of the native valve. Expandable sealing ring 120 may be constructed of materials such as, but not limited to stainless steel, Nitinol, cobalt-chromium alloys (e.g., L605), nickel-cobalt-chromium alloys (e.g., MP35N®) or other materials suitable for the purposes described herein. Expandable sealing ring 120 defines a passage 128, as shown in FIG. 3B. In another embodiment, shown in FIG. 3C, an expandable sealing ring 120′ may include a sealing material 129 disposed around an outer surface thereof. Sealing material 129 may be made of materials such as, but not limited to, nylon, polybutester, silk, polyester, flexible and impermeable materials such as PTFE, and other materials suitable for the purposes described herein. Further, other embodiments described below may also include such sealing materials.

In the embodiment of FIGS. 2A-2B, expandable sealing ring 120 is deployed at inflow portion 108 of valve component 102. In the embodiment of FIGS. 2A-2B, expandable sealing ring 120 is also disposed entirely within valve component 102, such that first end 124 and second end 126 are disposed within valve component 102. Although not shown, a single expandable sealing ring 120 may alternatively be disposed entirely within outflow portion 110 of valve component 102.

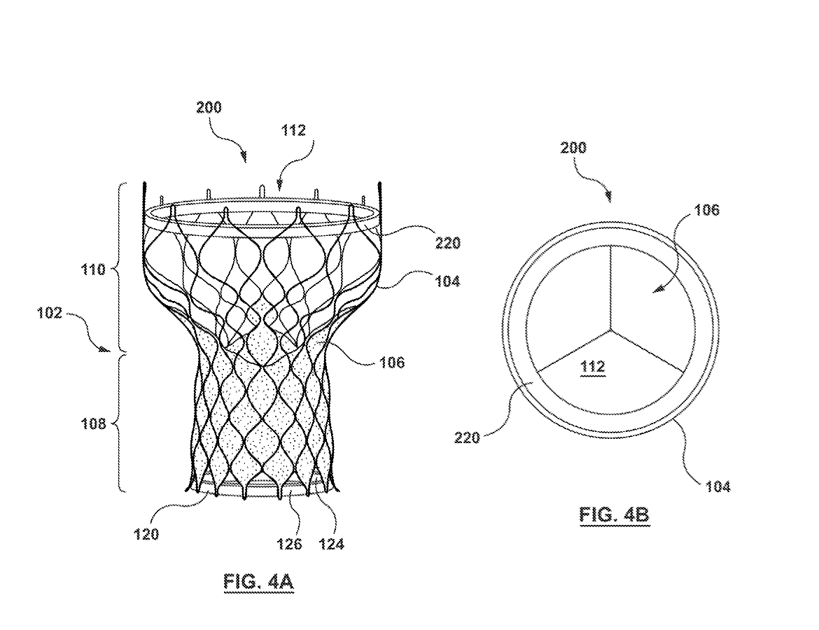

FIGS. 4A-4B show a sealing system 200 according to another embodiment hereof. Sealing system 200 includes a valve component 102 including a frame 104 and a prosthetic valve 106, as described above with respect to FIGS. 2A-2B. Sealing system 200 also includes an expandable sealing ring 120 disposed entirely within inflow portion 108 of valve component 102, as described above and shown in FIG. 2A. Sealing system further includes a second expandable sealing ring 220 disposed entirely within outflow portion 108 of valve component 102. Sealing system 200 uses the same reference numerals as sealing system 100 of FIGS. 2A-2B for items that are similar or identical to the embodiment of FIGS. 2A-2B.

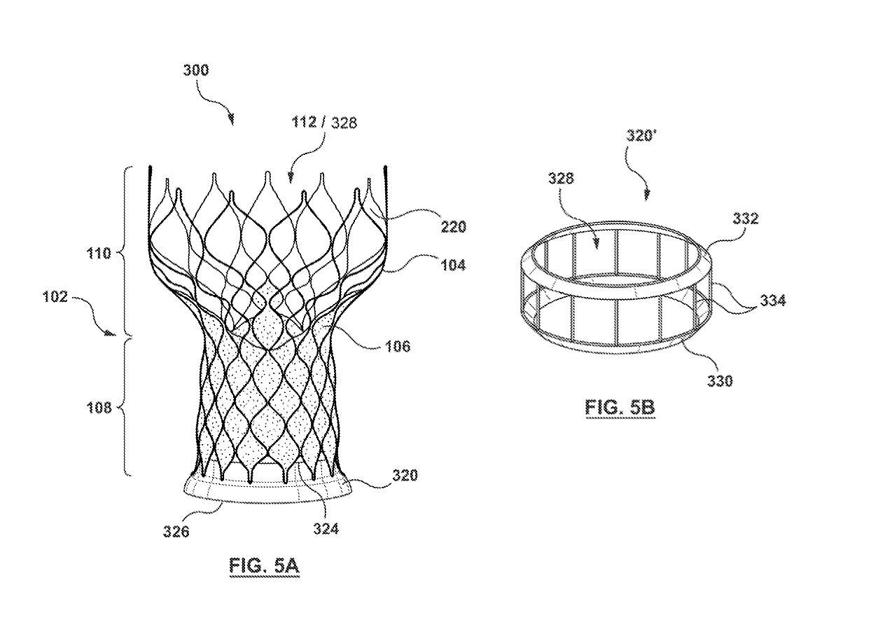

FIG. 5A show a sealing system 300 according to another embodiment hereof. Sealing system 300 includes a valve component 102 including a frame 104 and a prosthetic valve 106, as described above with respect to FIGS. 2A-2B. Sealing system 300 also includes an expandable sealing ring 320 partially disposed within valve component 102 such that a longitudinal first end 324 of expandable sealing ring 320 is disposed longitudinally outside of valve component 102 and longitudinal second end 326 of expandable sealing ring 320 is disposed longitudinally within valve component 102. In the embodiment shown FIG. 5A, expandable sealing ring 320 is deployed at inflow portion 108 of valve component 102. Expandable sealing ring 320 alternatively may be partially disposed at outflow portion 110 of valve component 102 such that first end 324 of expandable sealing ring 320 is disposed within valve component 102 and second end 326 is disposed longitudinally outside of valve component 102.

In an embodiment, expandable sealing ring 320 may simply be longitudinally longer than sealing ring 120 described above, as shown in FIG. 5A. In another embodiment shown in FIG. 5B, expandable sealing ring 320′ includes a first ring 330, a second ring 332, and longitudinal connectors 334 coupling first ring 330 and second ring 332 to each other. In such an embodiment, first ring 330 may be disposed longitudinally outside of valve component 102 and second ring 332 may be disposed longitudinally within valve component 102.

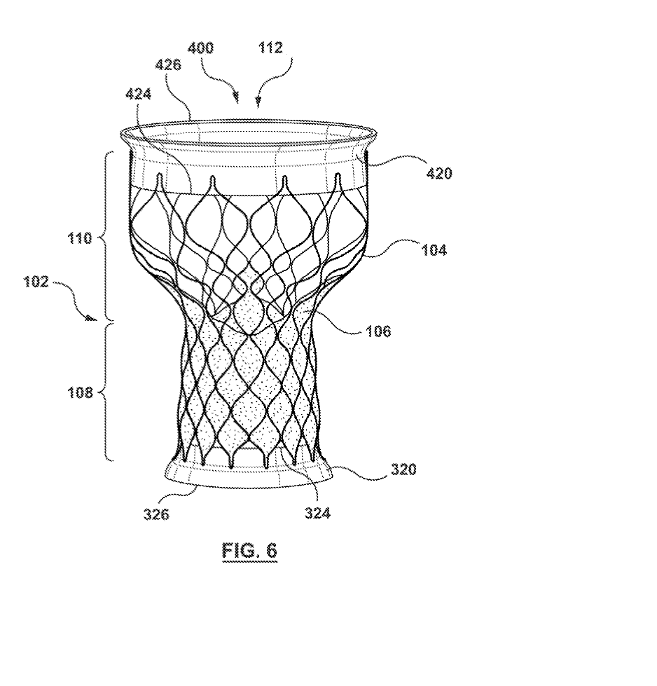

FIG. 6 shows a sealing system 400 according to another embodiment hereof. Sealing system 400 includes a valve component 102 including a frame 104 and a prosthetic valve 106, as described above with respect to FIGS. 2A-2B. Sealing system 400 also includes an expandable sealing ring 320 disposed partially within inflow portion 108 of valve component 102 and partially longitudinally outside of valve component 102, as described above and shown in FIG. 6A. Sealing system 400 further includes a second expandable sealing ring 420 disposed partially longitudinally within outflow portion 110 of valve component 102 and partially longitudinally outside of valve component 102, as shown in FIG. 6. Thus, a longitudinal first end 424 of expandable sealing ring 420 is disposed within outflow portion 110 of valve component 102, and a longitudinal second end 426 of expandable sealing ring 420 is disposed longitudinally downstream of outflow portion 1110.

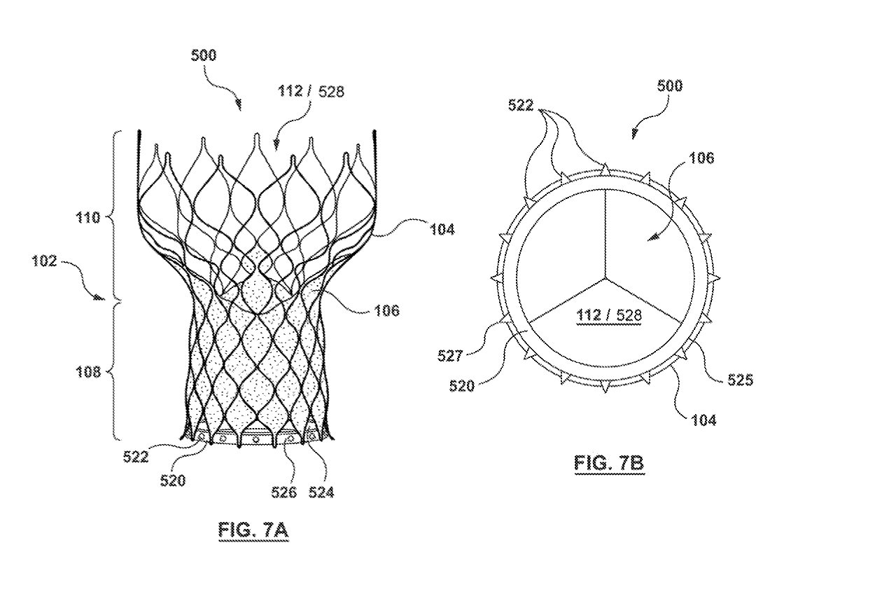

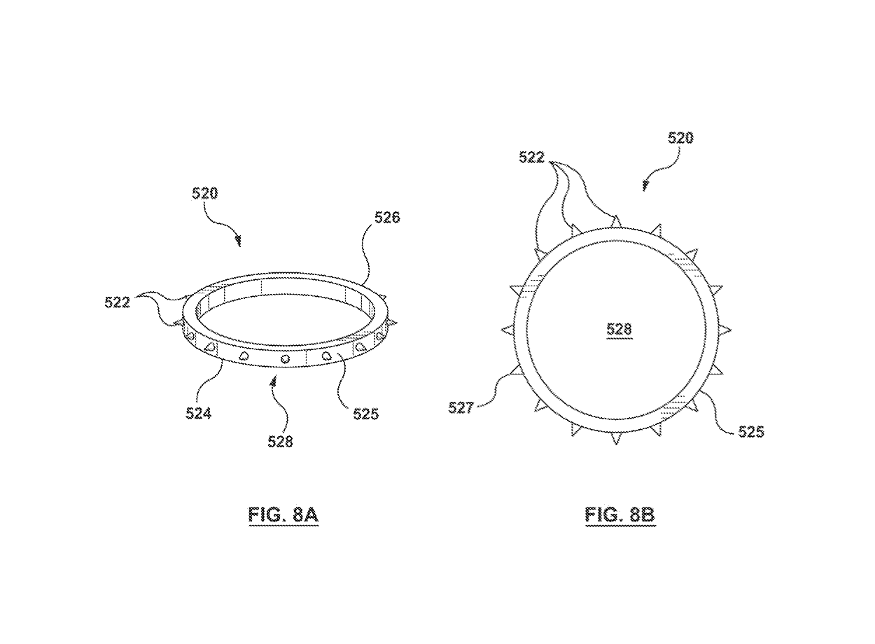

FIGS. 7A, 7B, 8A, and 8B show another embodiment of a sealing system 500. Sealing system 500 includes a valve component 102 and an expandable sealing ring 520. Valve component 102 includes a frame 104 and a prosthetic valve 106, as described above.

Expandable sealing ring 520 is similar to expandable sealing ring 120 described above. Accordingly, expandable sealing ring 500 is a generally annular ring defining a passage 528, as shown in FIGS. 8A-8B. Expandable sealing ring 520 has a longitudinal first end 524 and a longitudinal second end 526. Expandable sealing ring 520 further includes a plurality of protrusions 522 extending radially outward from an outer surface 525 of expandable sealing ring 520, as shown in FIGS. 7B, 8A, and 8B. Protrusions 522 may be formed as contiguous, integral components of expandable sealing ring 520, or may be coupled to expandable sealing ring 520 by methods such as, but not limited to, laser or ultrasonic welding, adhesives, or other methods suitable for the purposes disclosed herein. Protrusions 522 may include a sharp tip 527. Protrusions 522 may be configured such that with valve component 102 in its radially expanded deployed configuration, and expandable sealing ring 520 in an expanded configuration within valve component 102, protrusions 522 extend radially outward from outer surface 525 of expandable sealing ring 520 through frame 104 of valve component 102, and into a wall of a native valve. While a specific number and configuration of protrusions 522 are shown in FIGS. 7A, 7B, 8A, and 8B, this is not meant to limit the design and more or fewer protrusions 522 in various configurations may be utilized.

FIG. 7A shows expandable sealing ring 520 deployed entirely within valve component 102 at inflow portion 108 of valve component 102. However, as explained above, expandable sealing ring 520 may alternatively by deployed at outflow portion 110 of valve component 102, or at both inflow portion 108 and outflow portion 110. Further, expandable sealing ring 520 may be deployed entirely longitudinally within or only partially longitudinally within valve component 102, as described above.

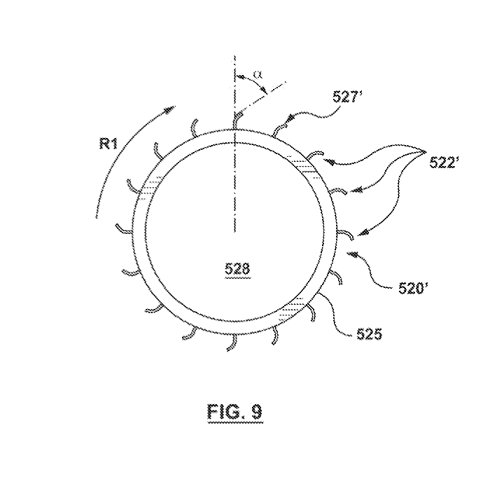

FIG. 9 shows another embodiment of an expandable sealing ring 520′ similar to expandable sealing ring 520. Expandable sealing ring 520′ differs from expandable sealing ring 520 in that protrusions 522′ of expandable sealing ring 520′ extend at an angle a relative to the radial direction. Angle a may be in the range of 15 to 50 degrees relative to the radial direction. Thus, when expandable sealing ring 520′ is disposed within inflow portion 108 or outflow portion 110 of valve component 102, and is rotated in a direction R1, as shown in FIG. 9, protrusions 522′ rotate in direction R1 and engage valve component 102 and the wall of the native valve.

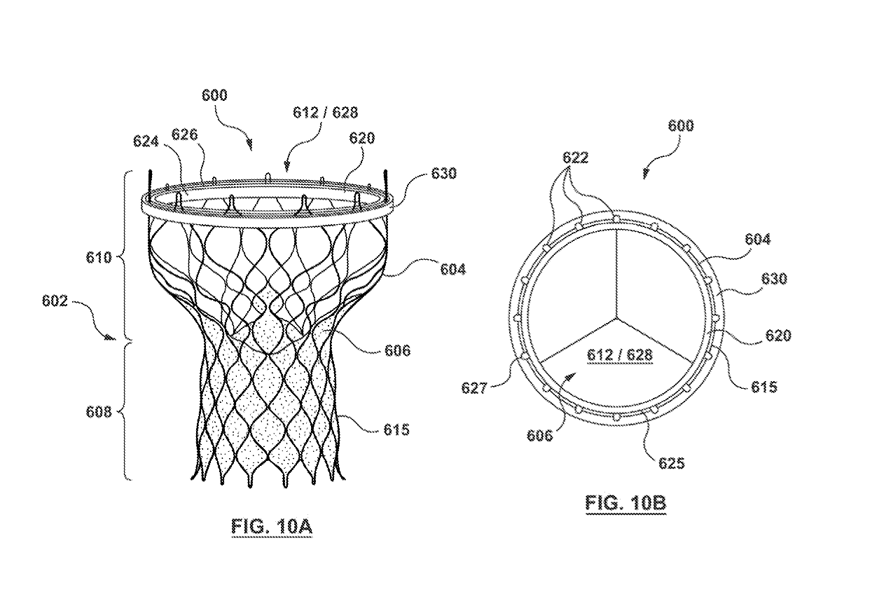

FIGS. 10A-10B show a sealing system 600 in accordance with another embodiment hereof. Sealing system 600 includes a valve component 602, an expandable sealing ring 620, and an outer ring 630. Valve component 602 is similar to the valve component 102 described above, incorporated into this embodiment by reference, and therefore will not be described in detail here. As with valve component 102, valve component 602 includes a frame 604 defining a central passage 612, and a prosthetic valve 606 coupled to frame 604 and disposed within central passage 612. Valve component 602 has an inflow portion 608 at a proximal end of valve component 602, and an outflow portion 610 at a distal end of valve component 602.

Expandable sealing ring 620 of the embodiment of FIGS. 10A-10B is the same as sealing ring 520 described above with respect to FIGS. 7A, 7B, 8A, and 8B. Accordingly, expandable sealing ring 620 is a generally annular ring defining a passage 628. Expandable sealing ring 620 has a longitudinal first end 624 and a longitudinal second end 626, and includes a plurality of protrusions 622 extending radially outward from an outer surface 625 of expandable sealing ring 620, as shown in FIG. 10B, and described above with respect to FIGS. 8A-8B. Protrusions 622 may include a sharp tip 627.

Outer ring 630 is a generally annular ring coupled to an outer surface 615 of valve component 602. Outer ring 630 is deployed with valve component 602. Outer ring 630 and valve component 602 are configured such that outer ring 630 is disposed between frame 604 and a wall of the native valve when valve component 602 is in the radially expanded deployed configuration. Outer ring 630 may be constructed of materials such as, but not limited to polyethylene terephthalate (PET), tissue (including porcine or bovine pericardium), or other biocompatible materials or other materials suitable for the purposes described herein. Outer ring 630 may be secured to frame 604 by methods such as, but not limited to, adhesives, sutures, laser or ultrasonic welding, or any other methods suitable for the purposes described herein.

In the embodiment shown in FIG. 10A, outer ring 630 is deployed radially outside of outflow portion 610 of valve component 602. Further, expandable sealing ring 620 is deployed radially inside of outflow portion 610 and is aligned with outer ring 630. Accordingly, when expandable sealing ring 620 is deployed (i.e., expanded radially outwardly) protrusions 622 extend through frame 604 at outflow portion 610 and into outer ring 630, as shown in FIG. 10B. Although FIGS. 10A-10B show a single expandable sealing ring 620 and a single outer ring 630 disposed at outflow portion 610 of valve component 602, expandable sealing ring 620 and outer ring 630 could alternatively be disposed at inflow portion 608, or there may be multiple expandable sealing rings 620 and outer rings 630 disposed at inflow portion 608, outflow portion 610, or both. Further, both expandable sealing ring 620 and outer ring 630 are shown in FIGS. 10A-10B as being disposed entirely longitudinally between ends of frame 604 (i.e., longitudinally within frame 604). However, expandable sealing ring 620 and outer ring 620 may be disposed partially between ends of frame 604 and partially longitudinally beyond or outside the ends of frame 604, as described above with respect to FIGS. 5A-5B and 6.

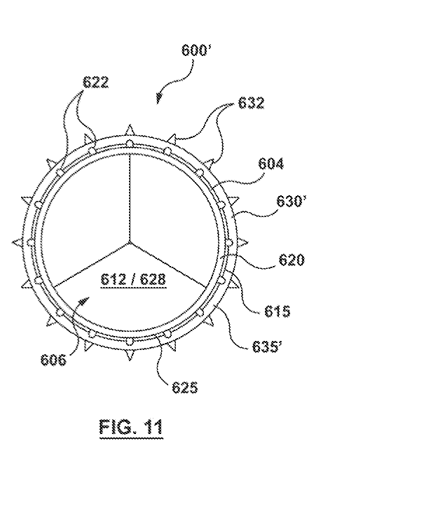

In another embodiment of a sealing system 600′, shown in FIG. 11, an outer ring 630′ may also include a plurality of protrusions 632 extending radially outward from an outer surface 635 of outer ring 630′. Protrusions 632 may be formed as a contiguous, integral component of outer ring 630′, or may be coupled to outer ring 630′ by methods such as, but not limited to laser or ultrasonic welding, adhesives, or other methods suitable for the purposes disclosed herein. Protrusions 632 may be configured such that protrusions 632 extend radially outward from outer surface 635 of outer ring 630′ and into the wall of the native valve. While a specific number and configuration of protrusions 632 are shown in FIG. 11, this is not meant to limit the design and more or fewer protrusions 632 in various configurations are envisioned based upon the application. Other details of sealing system 600′ of FIG. 11 are the same as sealing system 600 of FIGS. 10A-10B, and therefore are not described with respect to FIG. 11

While the various embodiments shown and described with respect to FIGS. 2A-11 provide possible configurations for sealing systems consistent with systems, devices, and methods of the present disclosure, they are not meant to limit the sealing systems to these configurations, and other materials, shapes, and combinations of expandable sealing rings and outer rings may be utilized. Further, each feature of each embodiment shown and/or described can be used in combination with the features of any other embodiment.

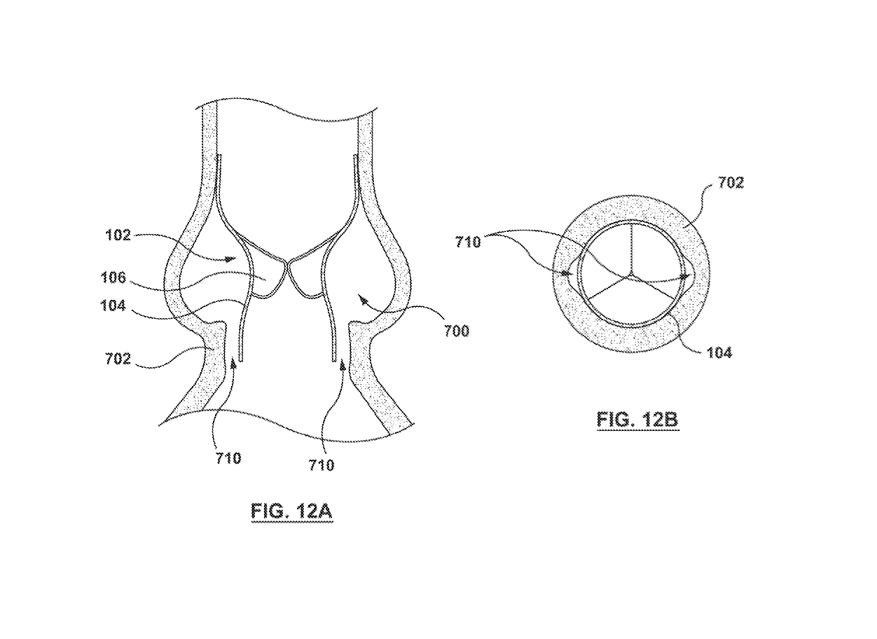

FIGS. 12A-14B schematically show an embodiment of a method of sealing a valve component to a wall of a native valve. The method of FIGS. 12A-14B can also be referred to as a method of remodeling an already deployed valve component or valvular prosthesis. FIGS. 12A-14B show the method using valve component 102, including frame 104 and prosthetic valve 106, and expandable sealing ring 120. However, this is merely exemplary, and the valve components and expandable sealing rings of other embodiments may be utilized. Further, in the embodiment of the method shown, expandable sealing ring 120 is disposed at inflow portion 108 of valve component 102. However, expandable sealing ring 120 may be disposed at outflow portion 110, or additional expandable sealing rings may be utilized and deployed at both inflow portion 108 and outflow portion 110, as described above.

FIGS. 12A-12B shows valve component 102 after it has been delivered and deployed at the site of a native valve 700. Methods and devices for delivering and deploying valve component 102 are known. Whether at the time of deployment or thereafter, and due to various factors, such as the misshapen nature or heavy calcification of walls of the native valve, valve component 102 is not 100% coapted to the wall of the native valve 700. As a result, voids 710 are present, which may result in paravalvular leakage (PVL).

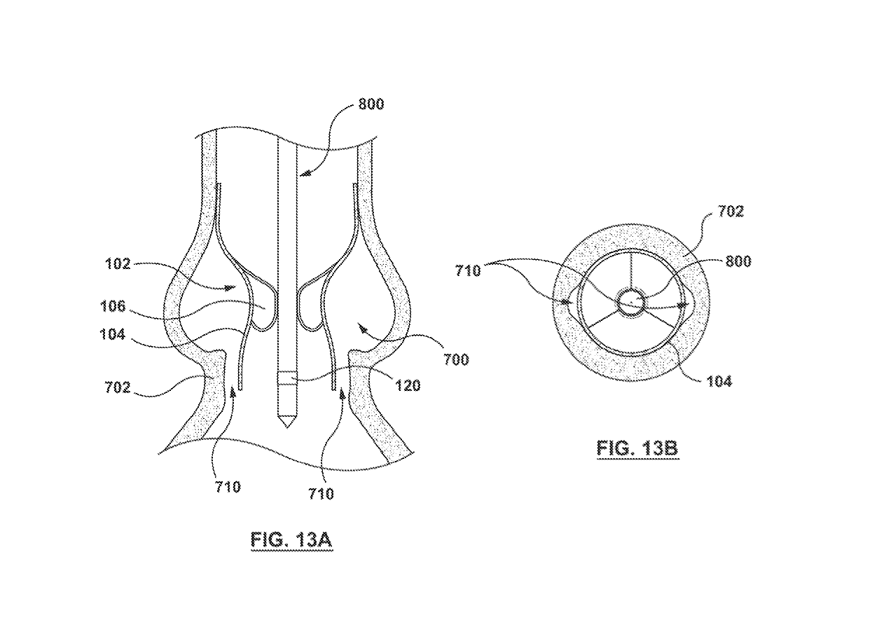

A delivery device 800 with a sealing ring 120 in a radially compressed configuration therein, is advanced through the patient's vasculature and is positioned within valve component 102, with valve component 102 in a radially expanded configuration, using established percutaneous transcatheter procedures, as shown in FIGS. 13A-13B.

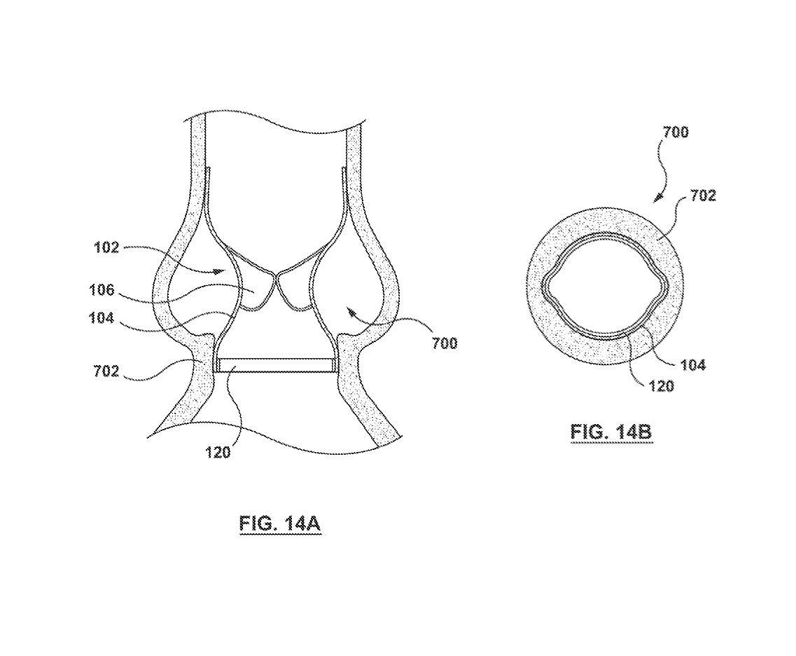

Expandable sealing ring 120 is deployed from delivery device 800 using known percutaneous transcatheter procedures, as shown in FIGS. 14A-14B. For example, and not by way of limitation, if expandable sealing ring 120 is self-expanding, expandable sealing ring 120 may be radially compressed in a sheath of delivery system 800 for delivery to the native valve 700. Once at the desired location, the sheath is retracted proximally, thereby enabling expandable sealing ring 120 to self-expand to its natural or pre-set expanded configuration. As expandable sealing ring 120 radially expands, expandable sealing ring 120 forces frame 104 of valve component 102 against the wall of the native valve 700, as shown in FIGS. 14A-14B. In the embodiment shown, the wall of the native valve is the aortic annulus 702 because the native valve is the aortic valve and the paravalvular leakage was determined to be cause at the inflow portion of valve component 102. However, expandable sealing ring 120 may be disposed in other portions of valve component 102 such that expansion of sealing ring 120 forces frame 104 against other walls of the native valve, as explained above. Further, if the expandable sealing ring 120 is balloon expandable or otherwise mechanically expandable, expandable sealing ring may be mounted on a balloon of a delivery system or coupled to a mechanical expansion mechanism. When the delivery system is at the desired location, the balloon or mechanical expansion mechanism is expanded, thereby expanding expandable sealing ring 120.

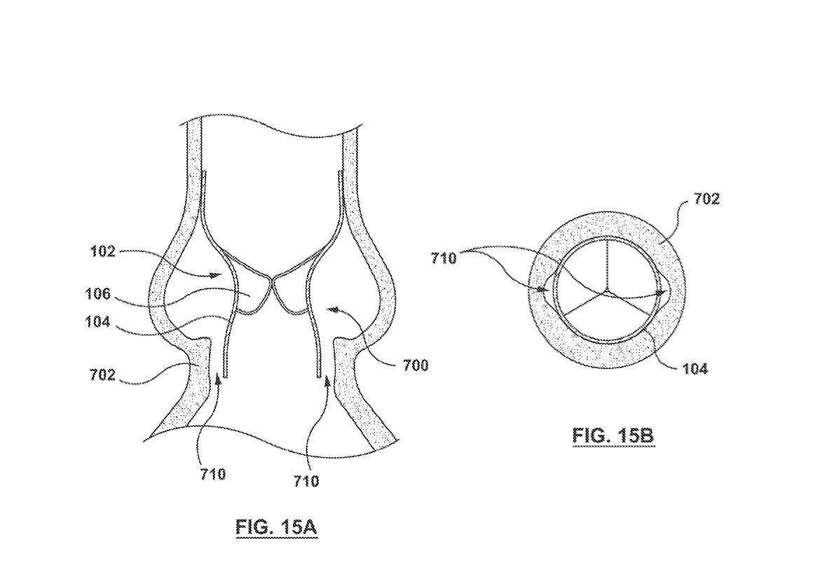

FIGS. 15A-17B show another embodiment of a method of sealing a valve component to a wall of a native valve. The method of FIGS. 15A-17B can also be referred to as a method of remodeling an already deployed valve component or valvular prosthesis. FIGS. 15A-17B show the method using expandable sealing ring 520 and valve component 102 of FIGS. 7A-8B. However, other embodiments as described above, in particular expandable sealing ring 520′ of FIG. 9, may also be used. Further, in the embodiment of the method shown, expandable sealing ring 520 is disposed at inflow portion 108 of valve component 102. However, expandable sealing ring 520 may be disposed at outflow portion 110, or additional sealing rings may be utilized and deployed at both inflow portion 108 and outflow portion 110, as described above.

FIGS. 15A-15B show valve component 102 after it has been delivered and deployed at the site of a native valve 700. Methods and devices for delivering and deploying valve component 102 are known. Whether at the time of deployment or thereafter, and due to various factors, such as the misshapen nature or heavy calcification of walls of the native valve, valve component 102 is not 100% coapted to the wall of the native valve 700. As a result, voids 710 are present, which may result in paravalvular leakage (PVL)

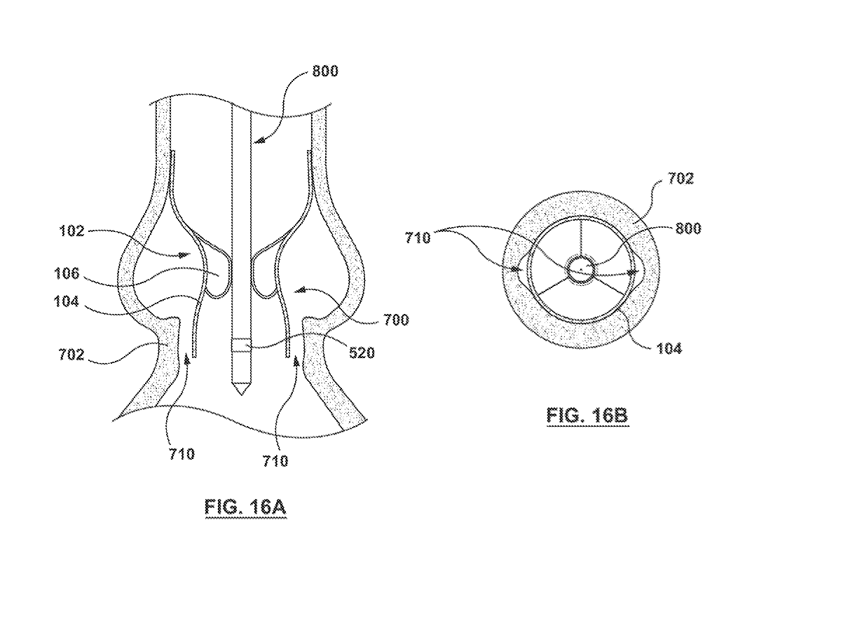

As shown in FIG. 16A-16B, a delivery device 800 with a sealing ring 520 in a radially compressed configuration therein, is advanced through the patient's vasculature and positioned within valve component 102, with valve component 102 in a radially expanded configuration, using established percutaneous transcatheter procedures

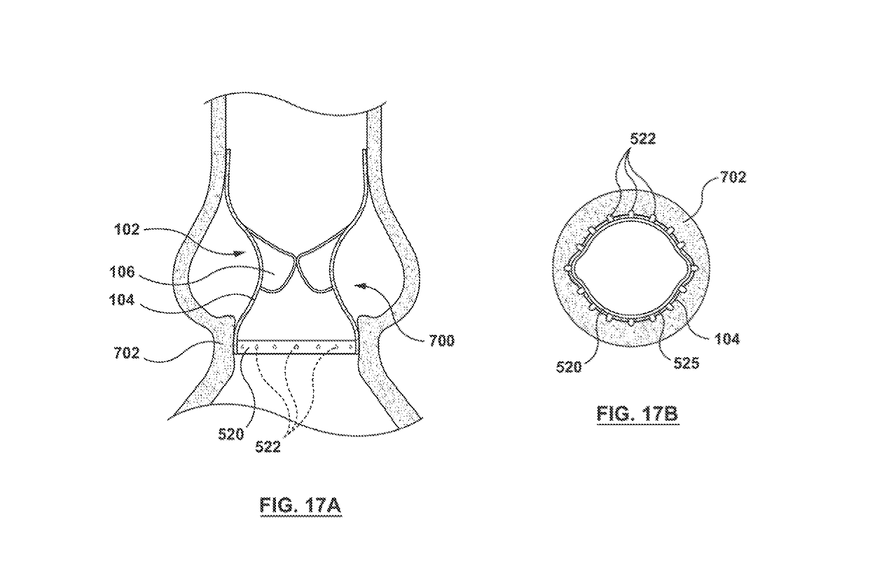

Expandable sealing ring 520 is deployed from delivery device 800 using known percutaneous transcatheter procedures, as shown in FIGS. 17A-17B. For example, and not by way of limitation, if expandable sealing ring 520 is self-expanding, expandable sealing ring 520 may be radially compressed in a sheath of delivery system 800 for delivery to the native valve 700. Once at the desired location, the sheath is retracted proximally, thereby enabling expandable sealing ring 520 to self-expand to its natural or pre-set expanded configuration. As expandable sealing ring 520 radially expands, expandable sealing ring 520 forces frame 104 of valve component 102 against the wall of the native valve 700, as shown in FIGS. 17A-17B. Further, as explained above, expandable sealing ring 520 includes protrusions 522 extending radially outward from outer surface 525. Therefore, as expandable sealing ring 520 radially expands, protrusions 522 extend through frame 104 of valve component 102 and engage the wall of the native valve 700, as shown in FIGS. 17A-17B.

In the embodiment shown, the wall of the native valve is the aortic annulus 702 because the native valve is the aortic valve and the paravalvular leakage was determined to be caused at the inflow portion of valve component 102. However, expandable sealing ring 520 may be disposed in other portions of valve component 102 such that expansion of sealing ring 520 forces valve component 102 against other walls of the native valve, as explained above. Further, if the expandable sealing ring 520 is balloon expandable or otherwise mechanically expandable, expandable sealing ring 520 may be mounted on a balloon of a delivery system or coupled to a mechanical expansion mechanism. When the delivery system is at the desired location, the balloon or mechanical expansion mechanism is expanded, thereby expanding expandable sealing ring 520.

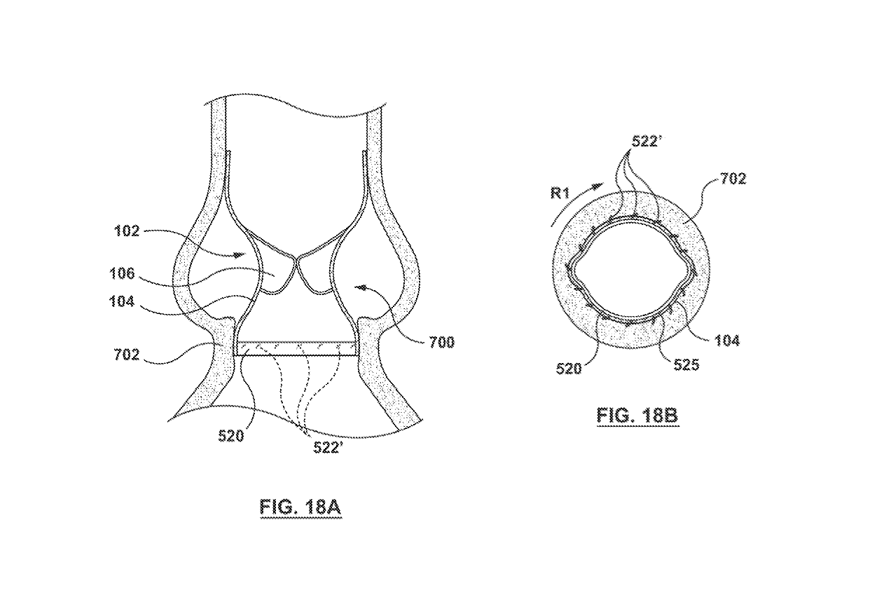

In another embodiment of the method, expandable sealing ring 520′ is utilized, with the plurality of protrusions 522′ which extend radially outward at an angle α relative to the radial direction. In this method, after sealing ring 520′ is expanded radially outward to the radially expanded configuration, sealing ring 520′ is rotated in a direction R1 such that protrusions 522′ engage valve component 102 and the wall of the native valve 700, as shown in FIGS. 18A-18B.

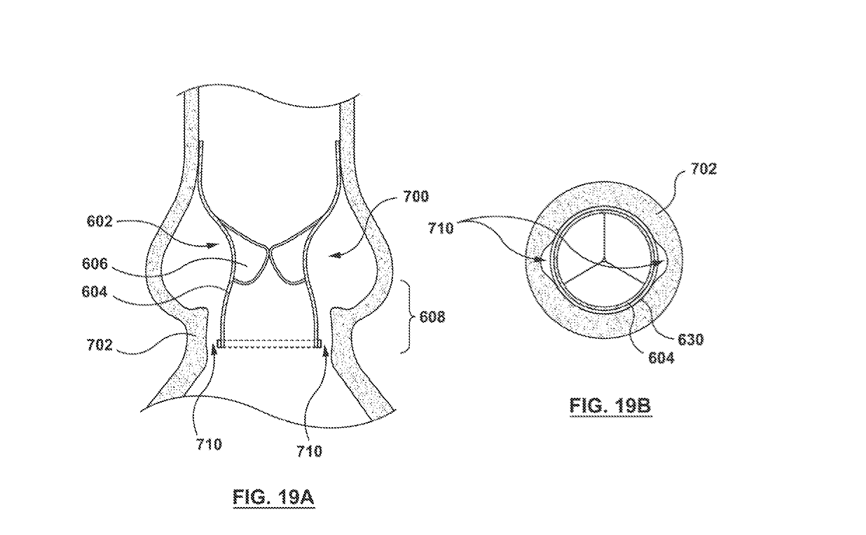

FIGS. 19A-21B schematically show an embodiment of a method of sealing a valve component to a wall of a native valve utilizing the sealing system 600 of FIGS. 10A-10B. The method of FIGS. 19A-21B can also be referred to as a method of remodeling an already deployed valve component or valvular prosthesis. FIGS. 19A-19B show valve component 602, including frame 604, prosthetic valve 606, and outer ring 630, after it has been delivered and deployed at the site of a native valve 700. Outer ring 630 is disposed between the wall of the native valve 700 and frame 604 with frame 604 in a radially expanded configuration. Methods and devices for delivering and deploying valve component 602 are known. Whether at the time of deployment or thereafter, and due to various factors, such as the misshapen nature or heavy calcification of the wall of the native valve 700, valve component 602 is not 100% coapted to wall of the native valve 700. As a result, voids 710 are present, which may result in paravalvular leakage (PVL).

A delivery device 800 with a sealing ring 620 in a radially compressed configuration is advanced through the patient's vasculature and positioned within frame 604, with frame 604 in the radially expanded configuration, using known percutaneous transcatheter procedures. Sealing ring 620 includes a plurality of protrusions 622 extending radially outward from an outer surface 625 of sealing ring 620. Delivery device 800 is advanced within frame 604 such that sealing ring 620 is aligned with outer ring 630.

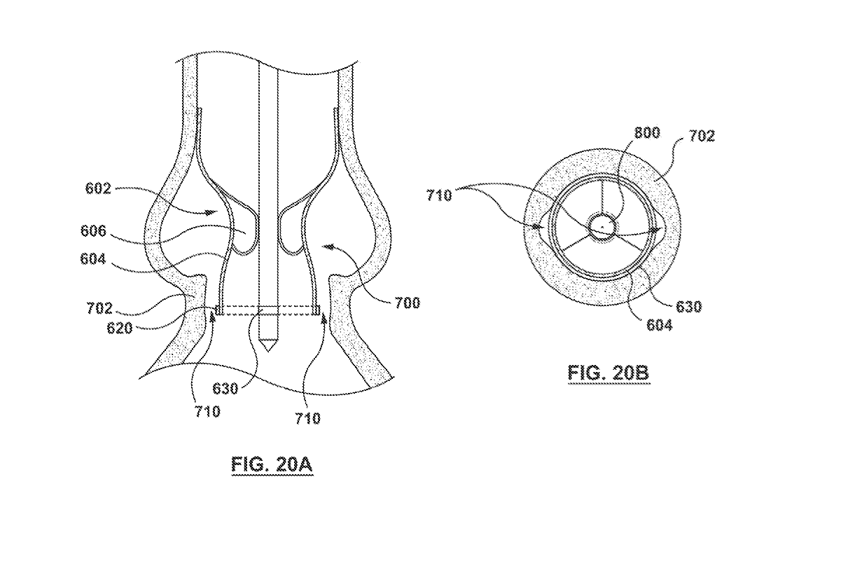

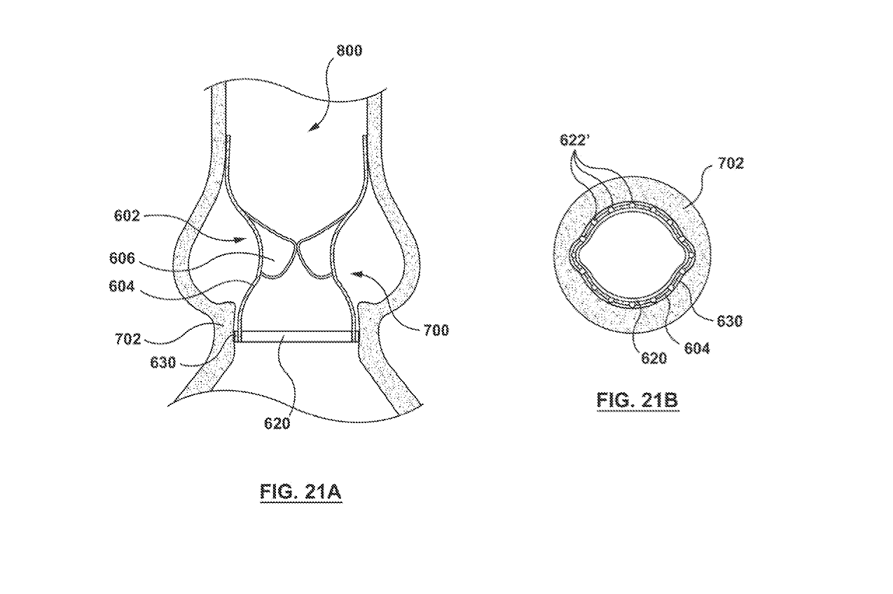

Expandable sealing ring 620 is deployed from delivery device 800 using known percutaneous transcatheter procedures, as shown in FIG. 20A-20B. For example, and not by way of limitation, if expandable sealing ring 620 is self-expanding, expandable sealing ring 620 may be radially compressed in a sheath of delivery system 800 for delivery to the native valve 700. Once at the desired location, the sheath is retracted proximally, thereby enabling expandable sealing ring 620 to self-expand to its natural or pre-set expanded configuration. As expandable sealing ring 520 radially expands, expandable sealing ring 620 forces frame 604 of valve component 602 radially outward towards the wall of the native valve 700. Further, protrusions 622 of sealing ring 620 expand radially outward with sealing ring 620 and into outer ring 630, as shown in FIGS. 21A-21B.

In the method shown in FIGS. 19A-21B, outer ring 630 is shown without projections. The method described in FIGS. 19A-21B may also be used with the outer ring 630′ described with respect to FIG. 11. Using outer ring 630′, when expandable sealing ring 620 expanded radially outwardly, expandable sealing ring forces fame 604 and outer ring 630′ radially outwardly, and protrusions 632 of outer ring 630′ are forced radially outward into the wall of the native valve 700, as shown in FIG. 11.

Similar methods as previously described may be used for various embodiments and configurations of the present disclosure including, but not limited to a plurality of sealing rings, various configurations of protrusions on sealing rings and outer rings, and varied positioning of sealing rings and outer rings at both inflow and/or outflow portions of the valve component, as described herein.

While only some embodiments and methods have been described herein, it should be understood that it has been presented by way of illustration and example only, and not limitation. Various changes in form and detail can be made therein without departing from the spirit and scope of the invention, and each feature of each embodiment discussed herein, and of each reference cited herein, can be used in combination with the features of any other embodiment. All patents and publications discussed herein are incorporated by reference herein in their entirety.

What is claimed is:

1. A sealing system comprising:
a valve component including a frame and a prosthetic valve coupled to the frame, the valve component having a radially expanded deployed configuration, an inflow portion and an outflow portion, the frame defining a central passage, the prosthetic valve disposed within the central passage of the frame; and
an expandable sealing ring configured to be inserted in a compressed configuration within the central passage with the valve component in the radially expanded deployed configuration, the expandable sealing ring configured to be radially expanded to apply a radially outward force to the valve component to expand the frame.

2. The sealing system of claim 1, wherein the expandable sealing ring comprises a plurality of expandable sealing rings.

3. The sealing system of claim 1, wherein the expandable sealing ring is disposed partially within the valve component.

4. The sealing system of claim 1, wherein the expandable sealing ring is disposed entirely within the valve component.

5. The sealing system of claim 1, wherein the expandable sealing ring is disposed at the inflow portion of the valve component.

6. The sealing system of claim 1, wherein the expandable sealing ring is disposed at the outflow portion of the valve component.

7. The sealing system of claim 1, wherein the expandable sealing ring further comprises a plurality of protrusions on an outer surface of the expandable sealing ring, the protrusions extending radially outward.

8. The sealing system of claim 7, wherein the protrusions are angled such that when the expandable sealing ring is rotated in a first direction, the protrusions engage the valve component and a wall of the valve.

9. The sealing system of claim 1, further comprising an outer ring disposed between an outer surface of the frame and a wall of the native valve when the valve component is in the radially expanded deployed configuration, wherein the expandable sealing ring includes protrusions which couple the expandable sealing ring to the outer ring.

10. The sealing system of claim 9, wherein the outer ring includes a plurality of protrusions configured to embed in the native artery wall when the valve component is in the radially expanded deployed configuration.

11. The sealing system of claim 1, wherein the expandable sealing ring is selected from the group consisting of self-expanding, balloon expandable, and mechanically expandable.

12. A method of remodeling a valvular prosthesis, the valvular prosthesis a frame and a prosthetic valve coupled to the frame, the method comprising the steps of:
advancing an expandable ring in a radially compressed configuration to a location within the fame of the valvular prosthesis with the frame in a radially expanded configuration within a native valve; and
expanding the expandable ring to a radially expanded configuration such that the expandable ring forces the frame radially outwardly.

13. The method of claim 12, wherein the steps of advancing, positioning and expanding the expandable ring comprises advancing, positioning, and expanding a plurality of sealing rings.

14. The method of claim 12, wherein the step of advancing the expandable ring comprises advancing the sealing ring to a location such that the expandable ring is disposed only partially within the frame.

15. The method of claim 12, wherein the step of advancing the sealing ring comprises advancing the expandable ring to a location such that the expandable ring is disposed entirely within the valve component.

16. The method of claim 12, wherein the step of advancing the expandable ring comprises advancing the expandable ring to location at an inflow portion of the valvular prosthesis.

17. The method of claim 12, wherein the step of advancing the expandable ring comprises advancing the expandable ring to a location at an outflow portion of the valvular prosthesis.

18. The method of claim 12, wherein the expandable ring includes a plurality of protrusions on an outer surface of the expandable ring, and the step of expanding the expandable ring includes forcing the protrusions radially outward into the wall of the native valve.

19. The method of claim 18, wherein the protrusions are angled with respect to a radial direction of the expandable ring, further comprising the step of rotating the expandable ring in a first direction such that the protrusions engage the frame the wall of the native valve.

20. The method of claim 12, wherein the expandable ring includes a plurality of protrusions, and wherein the step of expanding the expandable ring includes forcing the protrusions radially outward into an outer ring disposed between an outer surface of the frame and the wall of the native valve.

21. The method of claim 12, wherein the step of expanding the expandable ring is selected from the group consisting of releasing the expandable ring such that the expandable ring self-expands, inflating a balloon to expand the expandable ring, and actuating a mechanical expansion mechanism to expand the expandable ring.
